(19) United States
(12) Patent Application Publication
(10) Pub. No.: US 2007/0021396 A1
(43) Pub. Date: Jan. 25, 2007

(54) Oral contraception with trimegestone

(76) Inventors: Oliver Gloger, Aachen (DE); Heinrich Kugelmann, Aachen (DE); Maria Popova, Aachen (DE)

Correspondence Address: PERMAN & GREEN, 425 POST ROAD, FAIRFIELD, CT, 06824, US

(21) Appl. No.: 11/348,545

(22) Filed: Feb. 6, 2006

(30) Foreign Application Priority Data
Jul. 20, 2005 (DE) 10 2005 034 498.4

(51) Int. Cl. A61K 31/57; A61K 31/56

(52) U.S. Cl. 514/170

(57) ABSTRACT

The invention relates to a method for contraception comprising the administration of trimegestone to a woman of child-bearing age on at least 21 successive days, beginning on day 1 of the menstrual cycle, wherein on at least one of the at least 21 successive days the daily dose of trimegestone is more than 500 μg.

### CROSS-REFERENCE TO RELATED APPLICATIONS

This application claims priority from German Patent Application No. 10 2005 034 498.4.

### BACKGROUND OF THE INVENTION

1. Field of the Invention

The invention relates to a method for contraception by the administration of trimegestone. The invention further relates to pharmaceutical compositions and dosage forms which contain trimegestone.

2. Background Art

Trimegestone (17β-[(S)-2-hydroxypropanoyl]-17α-methyl-estra-4,9-dien-3-one) is a known prior art gestagen. Reference may for example be made in this connection to EP-A 007 823. Combinations of trimegestone with oestrogens for contraception are described, for example, in WO 98/04246, WO 98/04265, WO 98/04268 and WO 98/04269. WO 01/37841 discloses the administration of trimegestone in combination with oestradiol for treating the symptoms of the menopause and for preventing post-menopausal osteoporosis.

The majority of commercially available oral contraceptive preparations comprise a gestagen in combination with an oestrogen as the hormonal active ingredients, with administration conventionally proceeding for 21-25 days in each 28-day menstrual cycle. Thereafter, either a placebo or nothing at all is administered for 3-7 days, so initiating withdrawal bleeding.

In addition to effective contraception, a contraceptive preparation should, on the one hand, provide good cycle control and exhibit no or only slight side-effects. Good cycle control is in particular also distinguished by the occurrence of the desired (withdrawal) bleeding, which may inter alia be characterised by
the time interval between cessation of administration of the active ingredient and the onset of bleeding,
the duration of bleeding,
the extent of bleeding and
the occurrence of intermenstrual bleeding (for example spotting or breakthrough bleeding).

Since the introduction of oral contraceptive preparations, research has primarily focussed on the development of preparations which minimise the potential side-effects without in so doing exhibiting a reduced contraceptive action or deviating from the natural menstrual cycle of 28 days. The first generation of oral contraceptive preparations contained more gestagen and oestrogen than would per se have been necessary in order to ensure effective contraception. Disadvantageous haemostatic and metabolic changes, clinical problems and side-effects were associated with these high-dose first generation preparations. In 1978, the WHO recommended that the pharmaceutical industry should in future develop preparations with the lowest possible content of gestagen and oestrogen.

At first, the content of oestrogen was reduced in combination preparations because it was assumed that the side-effects known at that time, in particular thrombo-embolic disorders, were attributable to oestrogen. However, as it became increasingly clear that the gestagen was also associated with specific side-effects, in particular with cardiovascular complications, the content of gestagen in the combination preparations was also reduced. It was also recognised that a balance between oestrogen and gestagen may be established in order to avoid disadvantageous effects on carbohydrate metabolism and lipid or lipoprotein levels. It was subsequently found that, at a comparatively low dose of both the oestrogen and the gestagen, there is a synergistic action which inhibits ovulation.

Numerous therapeutic approaches have been developed in order to achieve the goal, while retaining contraceptive activity, of good cycle control and minimising the side-effects of the overall dose of steroids. In this connection, the gestagen/oestrogen combination is administered either at a constant dose (monophasic) or in a bi- or multiphasic regimen.

WO 98/04269, for example, discloses a monophasic regimen and WO 98/04265, WO 98/04268 and WO 98/04246 disclose multiphasic regimens, with inter alia 40-500 μg of trimegestone being administered daily in combination with an oestrogen. While according to A. E. Schindler et al., Maturitas, 2003, 46, S1, 7-16 the ovulation inhibition dose of trimegestone is 0.5 mg per day p.o., according to WO 98/04269, WO 98/04265, WO 98/04268 and WO 98/04246 the administered daily dose of trimegestone is preferably in the range from 40 to 250 μg. However, it is at least doubtful whether a daily dose of e.g. 40 μg trimegestone is sufficient in order to provide and to maintain a reliable contraceptive effect.

An ever greater reduction in the quantity of active ingredient cannot continue ad infinitum and may sometimes also cause new problems.

Accordingly, the problem sometimes arises with a minimised quantity of active ingredient that effective contraception and a stable menstrual cycle are more highly dependent on administration proceeding at the correct time so that a maximally constant plasma concentration of the active ingredients in the blood is maintained. Any deviations from a regular administration regimen, i.e. deviations from taking each day at the same time, should then as far as possible be avoided.

Entirely regular administration is, however, difficult to guarantee for practical reasons. It is known, for example, that a not inconsiderable proportion of women occasionally forget to take the dose intended for a particular day and only catch up on the following day. It may also happen, that the intended dose is administered in the morning on one day and not until the evening on the following day. Similar problems may also arise if the woman vomits after having taken the contraceptive, but before the dose has been completely resorbed.

The consequent fluctuations in plasma concentration may, as a result of the low dose of the administered active ingredients, possibly fall to values below the minimum threshold concentration which would be necessary to ensure reliable contraception.

In such cases, the effectiveness of the contraception cannot always be guaranteed with a minimised active ingredient dose. Apart from failure of the contraceptive action, the fluctuations in plasma concentration may furthermore also result in premature onset of (withdrawal) bleeding (intermenstrual bleeding, for example as spotting or breakthrough bleeding).

It is furthermore known that the metabolisation of active ingredients in the body may vary between individuals, for example due to a genetic disposition. It is accordingly possible that a low dose of trimegestone may in some women result in a plasma concentration which is above the necessary minimum concentration, but in other women, due to faster metabolisation, a higher dose would be necessary in order to ensure effective contraception.

Apart from the effectiveness of contraception, the course of withdrawal bleeding also plays an important role. It is in principle desirable for bleeding to occur for only a short time and to be only slight in extent. This is desirable not only from the subjective standpoint of most women, but also on medical grounds. Short and light bleeding, for example, is associated with only slight loss of iron.

The object of the invention is to provide a contraceptive method which exhibits advantages over prior art methods. Apart from ensuring effective contraception, the method should ensure good cycle control and exhibit no or at most only slight side-effects, for example no disadvantageous effects on carbohydrate metabolism and lipid or lipoprotein levels. These properties should be relatively insensitive to irregularities in administration of the active ingredients and to interindividual variations.

This object is achieved by the subject matter of the claims.

### BRIEF SUMMARY OF THE INVENTION

It has been surprisingly found that, when trimegestone is administered in combination with an oestrogen for oral contraception, the ratio of gestagen to oestrogen may be varied within relatively broad limits thereby providing a reliable contraceptive effect without consequently giving rise to increased side-effects, such as for example disadvantageous effects on carbohydrate metabolism and lipid or lipoprotein levels. It has thus surprisingly been found that the dose of trimegestone may be increased within certain limits without simultaneously also having to increase the dose of the oestrogen in order to maintain the gestagen-oestrogen balance. In this way, side-effects which would otherwise accompany an elevated dose of the oestrogen are prevented.

### DETAILED DESCRIPTION OF THE INVENTION

The invention relates to a method for contraception comprising preferably oral administration of
trimegestone,
optionally in combination with at least one oestrogen, preferably ethinyloestradiol, and/or
optionally in combination with at least one further gestagen, and/or
optionally in combination with at least one further physiologically active substance,
to a woman of child-bearing age on at least 21, preferably 21 to 26, more preferably 22 to 25 and most preferably 23 or 24 successive days of a preferably 28-day menstrual cycle, beginning on day 1 of the menstrual cycle,
wherein on at least one, preferably at least on 2, more preferably at least on 5, still more preferably at least on 8, most preferably at least on 14 and in particular on all of the at least 21 successive days, the daily dose of trimegestone is more than 500 μg.

In preferred embodiments of the method according to the invention, on at least one of the at least 21, preferably 24 successive days the daily dose of trimegestone is in the range from more than 500 μg to less than 2,000 μg, or it is more than 2,000 μg. Preferably, on at least one of the at least 21, preferably 24 successive days the daily dose of trimegestone is
in the range from more than 500 μg to preferably less than 1,000 μg, preferably from 510 to 990 μg, more preferably from 525 to 975 μg, still more preferably from 550 to 950 μg, most preferably from 575 to 925 μg and in particular from 600 to 900 μg; or
in the range from ≧1,000 μg to preferably less than 2,000 μg, preferably from 1,010 to 1,990 μg, more preferably from 1,025 to 1,975 μg, still more preferably from 1,050 to 1,950 μg, most preferably from 1,075 to 1,925 μg and in particular from 1,100 to 1,900 μg; or
≧2,000 μg, preferably at least 2,100 μg, more preferably ≧2,500 μg, still more preferably at least 3,000 μg, most preferably at least 4,000 μg and in particular at least 5,000 μg.

In a preferred embodiment of the method according to the invention, trimegestone is administered in combination with at least one oestrogen at least on one, preferably on all of the at least 21, preferably 24 successive days. The oestrogen is preferably selected from the group consisting of chlorotrianisene, dienestrol, diethylstilbestrol, oestradiol (17β-oestradiol), oestriol, oestrone, ethinyloestradiol, hexoestrol, mestranol, methallenoestril, methyloestrenol, promestriene and conjugated oestrogens or the pharmaceutically acceptable esters thereof. Ethinyloestradiol or a combination of ethinyloestradiol and oestradiol (17β-oestradiol) are particularly preferred. Preferred pharmaceutically acceptable esters of the above listed oestrogens are acetates, propionates and valerates (for example oestradiol valerate).

The daily dose of the oestrogen preferably corresponds to an equivalent dose of 5.0 to 55 μg, more preferably of 10 to 50 μg, still more preferably of 15 to 48 μg, most preferably of 20 to 45 μg and in particular of 22 to 40 μg of ethinyloestradiol. If two or more oestrogens are used, the daily overall dose thereof preferably corresponds to the above-stated equivalent doses.

Particularly preferred embodiments of combinations of the daily dose X of trimegestone with the daily doses Y of ethinyloestradiol are summarised in the following table:

| Trimegestone | Ethinyloestradiol |
|---|---|
| 510 μg ≦ X ≦ 1,990 μg | 10 μg ≦ Y ≦ 50 μg |
| 510 μg ≦ X ≦ 1,900 μg | 12 μg ≦ Y ≦ 48 μg |
| 525 μg ≦ X ≦ 1,500 μg | 15 μg ≦ Y ≦ 45 μg |
| 525 μg ≦ X ≦ 975 μg | 18 μg ≦ Y ≦ 42 μg |
| 550 μg ≦ X ≦ 950 μg | 20 μg ≦ Y ≦ 40 μg |
| 2,000 μg < X | 10 μg ≦ Y ≦ 50 μg |
| 2,100 μg ≦ X | 10 μg ≦ Y ≦ 50 μg |
| 2,500 μg < X | 10 μg ≦ Y ≦ 50 μg |

Preferably, ethinyloestradiol is adminstered in a daily dose of 20±5 μg in combination with trimegestone, the daily dose of trimegestone being >500 μg, ≧625 μg, ≧750 μg, ≧875 μg, ≧1,000 μg, ≧1,125 μg, 2,250 μg, ≧1,375 μg, ≧1,500 μg, ≧1,625 μg, ≧1,750 μg, ≧1,875 μg, ≧2,000 μg, ≧2,125 μg, ≧2,250 μg, ≧2,375 μg, ≧2,500 μg, ≧2,625 μg, ≧2,750 μg, ≧2,875 μg, ≧3,000 μg, ≧3,125 μg, ≧3,250 μg, ≧3,375 μg, ≧3,500 μg, ≧3,625 μg, ≧3,750 μg, ≧3,875 μg, ≧4,000 μg, ≧4,125 μg, ≧4,250 μg, ≧4,375 μg, ≧4,500 μg, ≧4,625 μg, ≧4,750 μg, ≧4,875 μg or ≧5,000 μg.

In a particularly preferred embodiment, on at least one, preferably on all, of the at least 21, preferably 24 successive days
ethinyloestradiol is administered in a daily dose of 1.0 to 55 μg, preferably 20±5 μg, and/or
oestradiol (17β-oestradiol) is administered in a daily dose of 1,000 to 10,000 μg.

In another particularly preferred embodiment on at least one, preferably on all, of the at least 21, preferably 24 successive days trimegestone is administered
either not together with oestradiol (17β-oestradiol)
or together with a combination of oestradiol (17β-oestradiol) and ethinyloestradiol.

According to this embodiment, oestradiol (17β-oestradiol) is thus preferably only administered when ethinyloestradiol is also administered.

In a particularly preferred embodiment of the method according to the invention, on none of the at least 21, preferably 24 successive days is an oestrogen administered without trimegestone being administered.

Particularly preferred embodiments of combinations of the daily dose X of trimegestone with the daily doses Y of ethinyloestradiol and the daily dose Z of oestradiol (17β-oestradiol) are summarised in the following table:

| Trimegestone | Ethinyloestradiol | Oestradiol |
|---|---|---|
| 510 μg ≦ X ≦ | 1.0 μg ≦ Y ≦ 10 μg | 1,000 μg ≦ Z ≦ 10,000 μg |
| 1,990 μg | | |
| 510 μg ≦ X ≦ | 2.0 μg ≦ Y ≦ 10 μg | 1,100 μg ≦ Z ≦ 9,000 μg |
| 1,900 μg | | |
| 525 μg ≦ X ≦ | 3.0 μg ≦ Y ≦ 9.5 μg | 1,200 μg ≦ Z ≦ 8,000 μg |
| 1,500 μg | | |
| 525 μg ≦ X ≦ | 4.0 μg ≦ Y ≦ 9.5 μg | 1,300 μg ≦ Z ≦ 7,000 μg |
| 975 μg | | |
| 550 μg ≦ X ≦ | 5.0 μg ≦ Y ≦ 9.0 μg | 1,400 μg ≦ Z ≦ 6,000 μg |
| 950 μg | | |
| 575 μg ≦ X ≦ | 6.0 μg ≦ Y ≦ 9.0 μg | 1,500 μg ≦ Z ≦ 5,000 μg |
| 925 μg | | |
| 2,000 μg < X | 1.0 μg ≦ Y ≦ 10 μg | 1,000 μg ≦ Z ≦ 10,000 μg |
| 2,100 μg ≦ X | 1.0 μg ≦ Y ≦ 10 μg | 1,000 μg ≦ Z ≦ 10,000 μg |
| 2,500 μg < X | 1.0 μg ≦ Y ≦ 10 μg | 1,000 μg ≦ Z ≦ 10,000 μg |

In a preferred embodiment of the method according to the invention, trimegestone is administered in combination with at least one further physiologically active substance at least on one, preferably on all of the at least 21, preferably 24 successive days. Preferably, said further physiologically active substance selected from the group consisting of folic acid, folinic acid, vitamin C, vitamin B preparations, iron(II) preparations, iron(III) preparations, calcium preparations and magnesium preparations.

Examples of vitamin B preparations are vitamin B₁ preparations, such as thiamine hydrochloride and thiamine nitrate; vitamin B₂ preparations, such as riboflavin and riboflavin-5′-phosphate; nicotinamid preparations; vitamin B₆ preparations, such as pyridoxine hydrochloride; panthotenic acid preparations, such as dexpanthenol; and vitamin B₁₂ preparations, such as cyanocobalamin and hydroxocobalamin acetate.

Examples of iron(II) preparations are iron(II) sulfate, iron(II) carbonate, iron(II) chloride, iron(II) tartrate, iron(II) gluconate, iron(II) aspartate, iron(II) glycine sulfate, iron(II) fumarate, iron(II) ascorbate, iron(II) iodate, iron(II) succinate and ammonium iron(II) sulfate.

Examples of iron(III) preparations are iron(III) sodium citrate, iron(III) oxide/sucrose complex, sodium feredetate, iron(III) hydroxide, dextriferron, iron(III) citrate, chondroitin sulfate/iron(III) complex, iron(III) acetyltransferrin, iron(III) protein succinylate and potassium/iron(III) phosphate/citrate complex.

Examples of calcium preparations are calcium carbonate, calcium citrate, calcium hydrogenphosphate, calcium phosphate, calcium aspartinate, calcium bisaspartate, calcium hydrogenaspartate, calcium gluconate, calcium lactate, calcium lactogluconate, calcium glucoheptonate, calcium acetate, calcium saccharate, calcium orotate and calcium lactobionate.

Examples of magnesium preparations are magnesium hydrogenaspartate, magnesium L-aspartate hydrochloride, magnesium oxide, magnesium hydrogenphosphate, magnesium citrate, magnesium hydrogencitrate, magnesium sulfate, magnesium L-hydrogenglutamate, magnesium D-gluconate, magnesium orotate, mgnesium adipate and magnesium nicotinate.

In a preferred embodiment of the method according to the invention, the daily dose of trimegestone is identical on each of the at least 21, more preferably at least 22, still more preferably at least 23, most preferably at least 24 and in particular at least 25 successive days (=monophasic regimen), wherein administration preferably proceeds in each case in combination with at least one oestrogen.

In another preferred embodiment of the method according to the invention, the at least 21, more preferably at least 22, still more preferably at least 23, most preferably at least 24 and in particular at least 25 successive days are divided into two, three or more groups of days, wherein the daily dose of trimegestone is identical on all the days within a group, but the daily dose of trimegestone is different on successive days of different groups (=multiphasic regimen), and wherein administration preferably proceeds in each case in combination with at least one oestrogen, preferably ethinyloestradiol. Preferred regimens are listed in the following table, the daily dose of trimegestone being A1, A2 or A3 and the daily dose of the at least one oestrogen, preferably ethinyloestradiol, being B:

| | | Number of phases | | | | | | | |
|---|---|---|---|---|---|---|---|---|---|
| | | 1 | 2 | 2 | 3 | 3 | 3 | 4 | 4 |
| Embodiment no. | | 1 | 2₁ | 2₂ | 3₁ | 3₂ | 3₃ | 4₁ | 4₂ |
| Total duration | | 21-25 | 21-25 | 21-25 | 21-25 | 21-25 | 21-25 | 21-25 | 21-25 |
| [days of 28] | | | | | | | | | |
| 1 | Duration [days] | 21-25 | 7-13 | 7-13 | 3-8 | 3-8 | 3-8 | 3-8 | 3-8 |
| | Trimegestone dose | A1 | A2 | A1 | A2 | A2 | A1 | A2 | A2 |
| | Oestrogen dose | B | B | B | B | B | B | B | B |
| | (equivalent dose to | | | | | | | | |
| | ethinyloestradiol) | | | | | | | | |
| 2 | Duration [days] | | 12-18 | 12-18 | 4-15 | 4-15 | 4-15 | 4-15 | 4-15 |
| | Trimegestone dose | | A1 | A2 | A2 | A1 | A2 | A1 | A2 |
| | Oestrogen dose | | B | B | B | B | B | B | B |
| | (equivalent dose to | | | | | | | | |
| | ethinyloestradiol) | | | | | | | | |
| 3 | Duration [days] | | | | 4-15 | 4-15 | 4-15 | 4-15 | 4-15 |
| | Trimegestone dose | | | | A1 | A2 | A2 | A2 | A1 |
| | Oestrogen dose | | | | B | B | B | B | B |
| | (equivalent dose to | | | | | | | | |
| | ethinyloestradiol) | | | | | | | | |
| 4 | Duration [days] | | | | | | | 2-5 | 2-5 |
| | Trimegestone dose | | | | | | | A3 | A3 |
| | Oestrogen dose | | | | | | | B | B |
| | (equivalent dose to | | | | | | | | |
| | ethinyloestradiol) | | | | | | | | |

The particular ranges of values of the doses for the particular combinations of A1, A2, A3 and B for each one of these embodiments no. 1, 2₁, 2₂, 3₁, 3₂, 3₃, 4₁ and 4₂ may be found in the following tables a, b, c and d, the dose B of the at least one oestrogen being stated as the equivalent dose to ethinyloestradiol:

| a | a | |
|---|---|---|
| A1 | >500 | μg |
| A2 | ≧40 | μg |
| A3 | ≧0 | μg |
| B | 5.0-55 | μg |

or

| b | b1 | | b2 | | b3 | | b4 | |
|---|---|---|---|---|---|---|---|---|
| | preferably | | more | | still more | | in particular | |
| | | | preferably | | preferably | | | |
| A1 | 510-990 | μg | 525-975 | μg | 550-950 | μg | 550-750 | μg |
| A2 | 40-990 | μg | 40-750 | μg | 120-750 | μg | 260-500 | μg |
| A3 | 0-990 | μg | 0-750 | μg | 0-500 | μg | 260-500 | μg |
| B | 5.0-55 | μg | 10-50 | μg | 20-45 | μg | 25-40 | μg |

or

| c | c1 | | c2 | | c3 | | c4 | |
|---|---|---|---|---|---|---|---|---|
| | preferably | | more | | still more | | in particular | |
| | | | preferably | | preferably | | | |
| A1 | 1,010-1,990 | μg | 1,025-1,975 | μg | 1,050-1,950 | μg | 1,050-1,750 | μg |
| A2 | 40-1,990 | μg | 40-750 | μg | 120-750 | μg | 260-500 | μg |
| A3 | 0-1,990 | μg | 0-750 | μg | 0-500 | μg | 260-500 | μg |
| B | 5.0-55 | μg | 7.5-50 | μg | 10-45 | μg | 15-40 | μg |

or

| d | d1 | | d2 | | d3 | | d4 | |
|---|---|---|---|---|---|---|---|---|
| | preferably | | more | | still more | | in particular | |
| | | | preferably | | preferably | | | |
| A1 | >2,000 | μg | >2,500 | μg | ≧3,000 | μg | ≧4,000 | μg |
| A2 | 40-5,000 | μg | 40-750 | μg | 120-750 | μg | 260-500 | μg |
| A3 | 0-5,000 | μg | 0-750 | μg | 0-500 | μg | 260-500 | μg |
| B | 5.0-55 | μg | 7.5-50 | μg | 10-45 | μg | 15-40 | μg |

Thus, when combining the embodiments no. 1, 2₁, 2₂, 3₁, 3₂, 3₃, 4₁ and 4₂ with any one of the doses a, b1, b2, b3, b4, c1, c2, c3, c4, d1, d2, d3 and d4, respectively, the following preferred embodiments may be individualized: 1^{a}, 2₁^{a}, 2₂^{a}, 3₁^{a}, 3₂^{a}, 3₃^{a}, 4₁^{a} and 4₂^{a}; 1^{b1}, 2₁^{b1}, 2₂^{b1}, 3₁^{b1}, 3₂^{b1}, 3₃^{b1}, 4₁^{b1} and 4₂^{b1}; 1^{b2}, 2₁^{b2}, 2₂^{b2}, 3₁^{b2}, 3₂^{b2}, 3₃^{b2}, 4₁^{b2} and 4₂^{b2}; 1^{b3}, 2₁^{b3}, 2₂^{b3}, 3₁^{b3}, 3₂^{b3}, 3₃^{b3}, 4₁^{b3} and 4₂^{b3}; 1^{b4}, 2₁^{b4}, 2₂^{b4}, 3₁^{b4}, 3₂^{b4}, 3₃^{b4}, 4₁^{b4} and 4₂^{b4}; 1^{c1}, 2₁^{c1}, 2₂^{c1}, 3₁^{c1}, 3₂^{c1}, 3₃^{c1}, 4₁^{c1} and 4₂^{c1}; 1^{c2}, 2₁^{c2}, 2₂^{c2}, 3₁^{c2}, 3₂^{c2}, 3₃^{c2}, 4₁^{c2} and 4₂^{c2}; 1^{c3}, 2₁^{c3}, 2₂^{c3}, 3₁^{c3}, 3₂^{c3}, 3₃^{c3}, 4₁^{d1} and 4₂^{c3}; 1^{c2}, 2₁^{c4}, 2₂^{c4}, 3₁^{c4}, 3₂^{c4}, 3₃^{c4}, 4₁^{c4} and 4₂^{c4}; 1^{d1}, 2₁^{d1}, 2₂^{d1}, 3₁^{d1}, 3₂^{d1}, 3₃^{d1}, 4₁^{d1} and 4₂^{d1}; 1^{d2}, 2₂^{d2}, 2₂^{d2}, 3₁^{d2}, 3₂^{d2}, 3₃^{d2}, 4₁^{d2} and 4₂^{d2}; 1^{d3}, 2₁^{d3}, 2₂^{d3}, 3₁^{d3}, 3₂^{d3}, 3₃^{d3}, 4₁^{d3} and 4₂^{d3}; and 1^{d4}, 2₁^{d4}, 2₂^{d4}, 3₁^{d4}, 3₂^{d}₄, 3₃^{d4}, 4₁^{d4} and 4₂^{d4}. In the preceding list the embodiment e.g. “3₂^{b2}” refers to the triphasic regimen “3₂”, wherein trimegestone and the oestrogen are administered in daily dosages according to table b, values “b2”.

The equivalent dose to ethinyloestradiol may be effected by an equivalent quantity of each suitable oestrogen, the quantity here being selected such that the oestrogenic activity corresponds to that which would be brought about by the administration of ethinyloestradiol in the stated quantity, ethinyloestradiol itself being the preferred oestrogen. Two or more different oestrogens, for example ethinyloestradiol in combination with oestradiol, may also be used in a quantity which corresponds overall to the stated equivalent dose. Suitable methods for determining the equivalent dose are known to the person skilled in the art. Trimegestone is preferably used in combination with ethinyloestradiol or in combination with ethinyloestradiol and oestradiol (17β-oestradiol).

In the bi-, tri- and tetraphasic regimens, the daily dose of trimegestone and of the oestrogen is in each case constant on all days within a phase and different on two successive days of different phases.

Particularly preferred regimens 1′, 2₁′, 2₂′, 3₁′, 3₂′, 3₃′, 4₁′ and 4₂′ may be found in the following table, according to which ethinyloestradiol is administered on 24 successive days in a daily dose of 20±5 μg in combination with trimegestone in daily doses A1, A2 and A3, respectively, as defined in tables a, b, c and d supra:

| | | Number of phases | | | | | | | |
|---|---|---|---|---|---|---|---|---|---|
| | | 1 | 2 | 2 | 3 | 3 | 3 | 4 | 4 |
| Embodiment no. | | 1′ | 2₁′ | 2₂′ | 3₁′ | 3₂′ | 3₃′ | 4₁′ | 4₂′ |
| Total duration | | 24 | 24 | 24 | 24 | 24 | 24 | 24 | 24 |
| [days of 28] | | | | | | | | | |
| 1 | Duration [days] | 24 | 7-13 | 7-13 | 3-8 | 3-8 | 3-8 | 3-8 | 3-8 |
| | Trimegestone dose | A1 | A2 | A1 | A2 | A2 | A1 | A2 | A2 |
| | ethinyloestradiol | 20 ± 5 | 20 ± 5 | 20 ± 5 | 20 ± 5 | 20 ± 5 | 20 ± 5 | 20 ± 5 | 20 ± 5 |
| | dose [μg] | | | | | | | | |
| 2 | Duration [days] | | 12-18 | 12-18 | 4-15 | 4-15 | 4-15 | 4-15 | 4-15 |
| | Trimegestone dose | | A1 | A2 | A2 | A1 | A2 | A1 | A2 |
| | ethinyloestradiol | | 20 ± 5 | 20 ± 5 | 20 ± 5 | 20 ± 5 | 20 ± 5 | 20 ± 5 | 20 ± 5 |
| | dose [μg] | | | | | | | | |
| 3 | Duration [days] | | | | 4-15 | 4-15 | 4-15 | 4-15 | 4-15 |
| | Trimegestone dose | | | | A1 | A2 | A2 | A2 | A1 |
| | ethinyloestradiol | | | | 20 ± 5 | 20 ± 5 | 20 ± 5 | 20 ± 5 | 20 ± 5 |
| | dose [μg] | | | | | | | | |
| 4 | Duration [days] | | | | | | | 2-5 | 2-5 |
| | Trimegestone dose | | | | | | | A3 | A3 |
| | ethinyloestradiol | | | | | | | 20 ± 5 | 20 ± 5 |
| | dose [μg] | | | | | | | | |

In a preferred embodiment of the method according to the invention, trimegestone is not administered on all the days of the preferably 28-day menstrual cycle. Instead, it is preferred that, on the days which follow the at least 21, preferably 24 successive days,
a placebo,
a pharmaceutically acceptable preparation containing iron,
a preparation containing folic acid, folinic acid and/or a salt thereof, or
a preparation containing an oestrogen, preferably ethinylestradiol, preferably in a daily dose corresponding to an equivalent dose of ≦10 μg of ethinyloestradiol, is administered;
or nothing at all is administered.

In this manner, it is ensured that the menstrual cycle is terminated by the withdrawal bleeding, such that a new menstrual cycle may begin. The menstrual cycle preferably lasts 28 days.

According to another preferred embodiment of the method according to the invention, it is, however, also possible for the menstrual cycle to be longer than 28 days. This may be achieved according to the invention, by the cessation of trimegestone (and optionally at least one oestrogen and/or at least one further gestagen) not occurring until a later point in time, such that the withdrawal bleeding also does not occur until a later point in time and thus the menstrual cycle also does not end until a later point in time. In this embodiment, trimegestone is preferably administered on more than 28 successive days.

In this embodiment, (uninterrupted) administration of trimegestone proceeds on at least 42 or 56, more preferably at least 63, still more preferably at least 84, most preferably at least 105, 112 or 120 and in particular at least 126, 140, 150, 189 or 365 successive days, such that it is not intended to initiate withdrawal bleeding within this period. According to the invention, the continuous period for which trimegestone may be administered daily may also be still longer. In principle, it is accordingly possible to administer trimegestone on all successive days over one or more years, without any withdrawal bleeding occurring.

In a preferred embodiment of the method according to the invention, trimegestone is administered in combination with at least one further gestagen at least on one of the at least 21, preferably 24 successive days. The further gestagen is here preferably selected from the group consisting of allyloestrenol, chlormadinone, danazol, demegestone, desogestrel, dienogest, drospirenone, dydrogesterone, ethisterone, etynodiol, gestodene, gestonorone, hydroxyprogesterone, levonorgestrel, lynoestrenol, medroxyprogesterone, medrogestone, megestrol, methyloestrenol, methylnortestosterone, nomegestrol, norethisterone, norethynodrel, norgestrel, norgestimate, progesterone, promegestone and tibolone, or the pharmaceutically acceptable esters thereof. Preferred pharmaceutically acceptable esters of the above listed gestagens are acetates (for example chlormadinone acetate, medroxyprogesterone acetate, megestrol acetate, norethisterone acetate), caproates (for example hydroxyprogesterone caproate) and enantates (for example norethisterone enantate).

The daily dose of the further gestagen preferably corresponds to an equivalent dose of 100 to 5,000 μg, more preferably of 250 to 4,000 μg, still more preferably of 500 to 3,500 μg, most preferably of 750 to 3,000 μg and in particular of 1,000 to 2,500 μg of chlormadinone acetate.

The method according to the invention is carried out for at least one menstrual cycle. The method according to the invention is preferably carried out for two or more, in particular for at least 3, 4, 5 or 6 successive menstrual cycles.

The present invention also relates to a, preferably solid, pharmaceutical composition comprising trimegestone in a quantity of more than 500 μg, preferably at least 600 μg, still more preferably at least 700 μg, most preferably at least 1,000 μg and in particular at least 1,200 μg, in combination with ethinyloestradiol, preferably in a quantity of 20±5 μg.

The present invention also relates to a, preferably solid, pharmaceutical composition comprising trimegestone in a quantity
of more than 500 μg and preferably less than 1,000 μg, preferably of 510 to 990 μg, more preferably of 525 to 975 μg, still more preferably of 550 to 950 μg, most preferably of 575 to 925 μg and in particular of 600 to 900 μg; or
of ≧1,000 μg and preferably less than 2,000 μg, preferably of 1,010 to 1,990 μg, more preferably of 1,025 to 1,975 μg, still more preferably of 1,050 to 1,950 μg, most preferably of 1,075 to 1,925 μg and in particular of 1,100 to 1,900 μg; or
of ≧2,000 μg, preferably at least 2,100 μg, more preferably more than 2,500 μg, still more preferably at least 3,000 μg, most preferably at least 4,000 μg and in particular at least 5,000 μg.

The present invention also relates to a pharmaceutical composition comprising trimegestone in a quantity of more than 500 μg, preferably of at least 750 μg, still more preferably of at least 1,000 μg, most preferably of at least 2,000 μg and in particular of at least 3,000 μg, in combination with ethinyloestradiol in a quantity of preferably at least 5 μg.

The pharmaceutical composition according to the invention is preferably formulated for oral administration. It preferably assumes the form of (film coated) tablets, sugar coated tablets or multiparticulate form, preferably the form of microtablets, microcapsules, micropellets, accretion pellets, granules, extrudates, spheroids, beads or pellets, which may optionally be packaged in capsules or press-moulded to form (film coated) tablets. Dry-compacted formulations are also possible.

The present invention also relates to a dosage form comprising the pharmaceutical composition as described above, preferably for once daily, preferably oral administration. The dosage form according to the invention comprises trimegestone in a quantity of more than 500 μg; preferably of at least 510 μg; more preferably of at least 525 μg, at least 1,000 μg, at least 1,500 μg or at least 2,000 μg; still more preferably of 550 to 950 μg; most preferably of 575 to 925 μg and in particular of 600 to 900 μg, wherein the dosage form is preferably selected from the group consisting of film coated tablets, sugar coated tablets and capsules. In a preferred embodiment of the dosage form it comprises trimegestone in a quantity of ≧1,000 μg and less than 2,000 μg or of ≧2,000 μg. The dosage form according to the invention may assume multiparticulate form, preferably the form of microtablets, microcapsules, micropellets, accretion pellets, granules, extrudates, spheroids, beads or pellets, optionally packaged in capsules or press-moulded to form (film coated) tablets. Dry-compacted formulations are also possible.

In a preferred embodiment, the dosage form according to the invention is selected from the group consisting of film coated tablets, sugar coated tablets and capsules and comprises the pharmaceutical composition according to the invention.

The preferred embodiments described below relate both to the pharmaceutical composition according to the invention and to the dosage form according to the invention.

The pharmaceutical composition or dosage form according to the invention preferably additionally contains at least one oestrogen, preferably ethinyloestradiol. The at least one oestrogen is here preferably selected from the group consisting of chlorotrianisene, dienestrol, diethylstilbestrol, oestradiol (17β-oestradiol), oestriol, oestrone, ethinyloestradioli hexoestrol, mestranol, methallenoestril, methyloestrenol, promestriene and conjugated oestrogens or the pharmaceutically acceptable esters thereof. Preferred pharmaceutically acceptable esters are valerates (for example oestradiol valerate).

The quantity of the oestrogen preferably corresponds to an equivalent dose of 5.0 to 55 μg, more preferably of 10 to 50 μg, still more preferably of 15 to 48 μg, most preferably of 20 to 45 μg and in particular of 22 to 40 μg of ethinyloestradiol, ethinyoestradiol itself being the preferred oestrogen. If two or more oestrogens are used, the overall quantity thereof preferably corresponds to the above-stated equivalent doses.

In a preferred embodiment of the pharmaceutical composition or dosage form according to the invention, said composition or dosage form contains
either no oestradiol (17β-oestradiol)
or oestradiol (17β-oestradiol) in combination with ethinyloestradiol.

In a preferred embodiment, the pharmaceutical composition or dosage form according to the invention additionally contains at least one further gestagen apart from trimegestone. The further gestagen is here preferably selected from the group consisting of allyloestrenol, chlormadinone, danazol, demegestone, desogestrel, dienogest, drospirenone, dydrogesterone, ethisterone, etynodiol, gestodene, gestonorone, hydroxyprogesterone, levonorgestrel, lynoestrenol, medroxyprogesterone, medrogestone, megestrol, methyloestrenol, methylnortestosterone, nomegestrol, norethisterone, norethynodrel, norgestrel, norgestimate, progesterone, promegestone and tibolone, or the pharmaceutically acceptable esters thereof. Preferred pharmaceutically acceptable esters are acetates (for example chlormadinone acetate, medroxyprogesterone acetate, megestrol acetate, norethisterone acetate), caproates (for example hydroxyprogesterone caproate) and enantates (for example norethisterone enantate).

The quantity of the further gestagen preferably corresponds to an equivalent dose of 100 to 5,000 μg, more preferably of 250 to 4,000 μg, still more preferably of 500 to 3,500 μg, most preferably of 750 to 3,000 μg and in particular of 1,000 to 2,500 μg of chlormadinone acetate.

If, apart from trimegestone, the pharmaceutical composition or dosage form according to the invention contains further active ingredients, in particular at least one oestrogen (such as ethinyloestradiol) and/or a further gestagen, these are preferably present as a mixture within the same administration unit. Such dosage forms may be produced with the assistance of conventional methods and auxiliary substances. Suitable auxiliary substances are known to the person skilled in the art. In this connection, reference may be made, for example, to H. P. Fiedler, Lexikon der Hilfsstoffe für Pharmazie, Kosmetik und angrenzende Gebiete, Editio Cantor Aulendorff, 2002; and R. C. Rowe et al., Handbook of Pharmaceutical Excipients, APhA Publications, 4^{th} edition, 2003 in their entirety.

Examples of auxiliary substances are salt formers, buffers, emulsifiers, solubilising agents (solubilisers), wetting agents, antifoaming agents, gel formers, thickeners, film formers, surfactants, binders, slip agents, lubricants, embedding agents, mould release agents, flow-control agents, disintegration accelerators (disintegrants), chelating agents, sorbents, fillers, pharmaceutical solvents, antioxidants (for example α-tocopherol), preservatives, plasticizers, flavour and odour correctives and colorants.

Examples of extenders are lactose, mannitol, calcium diphosphate, starch, microcrystalline cellulose, calcium carbonate (E170) and magnesium carbonate.

Examples of disintegration accelerators (disintegrants) are starch, for example maize starch, potato starch, crosslinked polyvinylpyrrolidone and low substituted sodium carboxymethylcellulose.

Examples of binders are starch (e.g. potato starch, maize starch), gelatin, polyvinylpyrrolidone, cellulose ethers, sugars, for example sucrose and glucose syrup.

Examples of slip agents are talcum, sodium stearyl fumarate, fatty acid esters and macrogol.

Examples of lubricants are stearic acid, magnesium stearate, calcium stearate and zinc stearate.

An Example of a flow-control agent is colloidal silicon dioxide.

Examples of pharmaceutical solvents are propylene glycol and glycerol.

One example of a surfactant is polyoxyethylene/sorbitan fatty acid ester (for example Polysorbate 80).

Examples of colorants are indigo carmine (E132), titanium dioxide (E171) and quinoline yellow (E104).

Examples of film formers are shellac, methylcellulose, hypromellose (hydroxypropyl-methylcellulose, HPMC), hydroxypropylcellulose, hydroxyethylcellulose, ethylcellulose, polyacrylates and polymethacrylates. Plasticizers, such as propylene glycol and/or polyethylene glycol may additionally be contained in the film coating composition.

Examples of embedding agents are carnauba wax, montan glycol wax, stearic/palmitic acid, glycerol trioleate and cetylstearyl alcohol.

Examples of chelating agents are citric acid, phenylalanine, sodium calcium edetate and disodium edetate (EDTA-Na₂).

Examples of preparations containing iron are iron(II) preparations, such as for example iron(II) sulfate, iron(II) carbonate, iron(II) chloride, iron(II) tartrate, iron(II) gluconate, iron(II) aspartate, iron(II) glycine sulfate, iron(II) fumarate, iron(II) ascorbate, iron(II) iodate, iron(II) succinate and ammonium iron(II) sulfate; and iron(III) preparations, such as for example iron(III) sodium citrate, iron(III) oxide/sucrose complex, sodium feredetate, iron(III) hydroxide, dextriferron, iron(III) citrate, chondroitin sulfate/iron(III) complex, iron(III) acetyltransferrin, iron(III) protein succinylate and potassium/iron(III) phosphate/citrate complex.

In a particularly preferred embodiment, a preparation containing iron is administered in combination with folic acid, folinic acid and/or a salt thereof. The following iron preparations are particularly suitable for this embodiment: iron/amino acid complex, iron(II) fumarate, iron(II) sulfate, dextriferron, ammonium iron(II) sulfate, iron(II) glycine sulfate and iron(II) gluconate. The folic acid and the folinic acid, respectively, is here preferably present in free form or as its calcium salt.

When folic acid, folinic acid and/or a salt thereof is administered, its daily dose is preferably within the range of from 0.1 to 7.5 mg, more preferably from 0.2 to 5.0 mg, still more preferably from 0.3 to 3.0 mg, most preferably from 0.4 to 2.5 mg and in particular from 0.5 to 2 mg.

Examples of particularly preferred auxiliary substances are talc, long chain fatty acids, magnesium stearate, stearic acid, calcium stearate, polyethylene glycol, palmitic acid, and hydrogenated vegetable oils, such as hydrogenated castor oil.

In a preferred embodiment, the pharmaceutical composition or dosage form according to the invention contains a buffer with a pH value in the range from 2.0 to 5.5. The buffer is preferably formed by a mixture of citric acid and disodium hydrogenphosphate.

In a preferred embodiment, the pharmaceutical composition or dosage form according to the invention contains a cyclodextrin, such as β-cyclodextrin or γ-cyclodextrin, preferably β-hydroxypropyl-cyclodextrin (β-HP). Preferably, the cyclodextrin forms a complex with trimegestone and/or an oestrogen, e.g. with ethinyloestradiol.

In a preferred embodiment, apart from trimegestone and optionally at least one oestrogen and/or at least one further gestagen, the pharmaceutical composition or dosage form according to the invention contains a further physiologically active substance, such as folic acid, folinic acid or a suitable derivative or salt, for example the calcium salt, vitamin C, vitamin B preparations, iron(II) preparations, iron(III) preparations, calcium preparations and magnesium preparations.

In a preferred embodiment, apart from trimegestone and optionally at least one oestrogen and/or at least one further gestagen, the pharmaceutical composition or dosage form according to the invention contains the following auxiliary substances in the following preferred quantities (percentages are relative to the total weight of the dosage form):

| | | | more | | in | |
|---|---|---|---|---|---|---|
| | preferably | | preferably | | particular | |
| Constituent | [wt. %] | | [wt. %] | | [wt. %] | |
| HPMC | 1.0 to | 7.5 | 2.5 to | 5.0 | 3.0 to | 5.0 |
| Titanium dioxide | 0.1 to | 2.0 | 0.5 to | 1.5 | 0.7 to | 1.2 |
| Starch | 10 to | 60 | 20 to | 40 | 25 to | 35 |
| Lactose monohydrate | 25 to | 80 | 40 to | 70 | 50 to | 65 |
| Stearic acid | 0.1 to | 2.5 | 0.2 to | 1.5 | 0.3 to | 1.0 |
| Talcum | 0.1 to | 5.0 | 0.5 to | 2.5 | 0.9 to | 1.5 |

In another preferred embodiment, apart from trimegestone and optionally at least one oestrogen and/or at least one further gestagen, the pharmaceutical composition or dosage form according to the invention contains the following auxiliary substances in the following preferred quantities (percentages are relative to the total weight of the dosage form):

| | | | more | | in | |
|---|---|---|---|---|---|---|
| | preferably | | preferably | | particular | |
| Constituent | [wt. %] | | [wt. %] | | [wt. %] | |
| PVP | 0.1 to | 10 | 0.5 to | 7.5 | 1.0 to | 5.0 |
| Stearic acid | 0 to | 7.5 | 0.1 to | 5.0 | 0.5 to | 2.0 |
| Starch | 1.0 to | 50 | 2.5 to | 25 | 5.0 to | 15 |
| Colloidal silicon dioxide | 0 to | 7.5 | 0.1 to | 5.0 | 0.5 to | 2.0 |
| α-Tocopherol | 0 to | 1.0 | 0.001 to | 0.5 | 0.05 to | 0.2 |
| Lactose monohydrate | 10 to | 95 | 25 to | 92 | 50 to | 90 |
| Magnesium stearate | 0 to | 1.0 | 0.001 to | 0.5 | 0.05 to | 0.2 |

The pharmaceutical composition or dosage form according to the invention may, for example, contain the following substances in the following preferred quantities:

| Constituent [mg] | 1-A | 1-B | 1-C | 1-D | 1-E | 1-F |
|---|---|---|---|---|---|---|
| Trimegestone | 0.525 | 0.575 | 0.600 | 0.750 | 1.100 | 1.500 |
| Ethinyloestradiol | 0.020 | 0.020 | 0.020 | 0.020 | 0.020 | 0.020 |
| PVP | 2.400 | 2.400 | 2.400 | 2.400 | 2.400 | 2.400 |
| Stearic acid | 0.800 | 0.800 | 0.800 | 0.800 | 0.800 | 0.800 |
| Starch | 8.000 | 8.000 | 8.000 | 8.000 | 8.000 | 8.000 |
| Colloidal silicon dioxide | 0.800 | 0.800 | 0.800 | 0.800 | 0.800 | 0.800 |
| α-Tocopherol | 0.080 | 0.080 | 0.080 | 0.080 | 0.080 | 0.080 |
| Lactose monohydrate | 67.295 | 67.245 | 67.220 | 67.070 | 66.720 | 66.320 |
| Magnesium stearate | 0.080 | 0.080 | 0.080 | 0.080 | 0.080 | 0.080 |
| Σ | 80.000 | 80.000 | 80.000 | 80.000 | 80.000 | 80.000 |

Film-coated tablets may, for example, have the following composition:

| Constituent [mg] | 2-A | 2-B | 2-C | 2-D | 2-E | 2-F |
|---|---|---|---|---|---|---|
| Trimegestone | 0.525 | 0.575 | 0.600 | 0.750 | 1.100 | 1.500 |
| Ethinyloestradiol | 0.020 | 0.020 | 0.020 | 0.020 | 0.020 | 0.020 |
| Potato Starch | 8.000 | 8.000 | 8.000 | 8.000 | 8.000 | 8.000 |
| Lactose monohydrate | 67.290 | 67.240 | 67.215 | 67.065 | 66.715 | 66.315 |
| Stearic acid | 0.800 | 0.800 | 0.800 | 0.800 | 0.800 | 0.800 |
| α-Tocopherol | 0.080 | 0.080 | 0.080 | 0.080 | 0.080 | 0.080 |
| Colloidal silicon dioxide | 0.800 | 0.800 | 0.800 | 0.800 | 0.800 | 0.800 |
| Povidone K30 | 2.400 | 2.400 | 2.400 | 2.400 | 2.400 | 2.400 |
| Quinoline Yellow E104 | 0.005 | 0.005 | 0.005 | 0.005 | 0.005 | 0.005 |
| Magnesium stearate | 0.080 | 0.080 | 0.080 | 0.080 | 0.080 | 0.080 |
| HPMC (film coating) | 0.750 | 0.750 | 0.750 | 0.750 | 0.750 | 0.750 |
| PEG 6000 | 0.220 | 0.220 | 0.220 | 0.220 | 0.220 | 0.220 |
| Propylene glycol | 0.030 | 0.030 | 0.030 | 0.030 | 0.030 | 0.030 |
| Σ | 81.000 | 81.000 | 81.000 | 81.000 | 81.000 | 81.000 |
| Constituent [mg] | 3-A | 3-B | 3-C | 3-D | 3-E | 3-F |
| Trimegestone | 0.525 | 0.575 | 0.600 | 0.750 | 1.100 | 1.500 |
| Ethinyloestradiol | 0.030 | 0.030 | 0.030 | 0.030 | 0.030 | 0.030 |
| Potato Starch | 8.000 | 8.000 | 8.000 | 8.000 | 8.000 | 8.000 |
| Lactose monohydrate | 67.285 | 67.235 | 67.210 | 67.060 | 66.710 | 66.310 |
| Stearic acid | 0.800 | 0.800 | 0.800 | 0.800 | 0.800 | 0.800 |
| α-Tocopherol | 0.080 | 0.080 | 0.080 | 0.080 | 0.080 | 0.080 |
| Colloidal silicon dioxide | 0.800 | 0.800 | 0.800 | 0.800 | 0.800 | 0.800 |
| Polyvidone K30 | 2.400 | 2.400 | 2.400 | 2.400 | 2.400 | 2.400 |
| Magnesium stearate | 0.080 | 0.080 | 0.080 | 0.080 | 0.080 | 0.080 |
| HPMC (film coating) | 0.750 | 0.750 | 0.750 | 0.750 | 0.750 | 0.750 |
| PEG 6000 | 0.220 | 0.220 | 0.220 | 0.220 | 0.220 | 0.220 |
| Propylene glycol | 0.030 | 0.030 | 0.030 | 0.030 | 0.030 | 0.030 |
| Σ | 81.000 | 81.000 | 81.000 | 81.000 | 81.000 | 81.000 |

The present invention also relates to a kit comprising at least one of the above-described dosage forms according to the invention.

The kit according to the invention is preferably designed for in each case once daily administration of the dosage forms contained therein.

The kit preferably comprises all the dosage forms containing trimegestone which are necessary for administering trimegestone for at least one menstrual cycle. The kit is preferably made up such that the above-described method for contraception according to the invention may be carried out without entailing the acquisition of further dosage forms containing trimegestone which are not contained in the kit. The kit preferably contains one dosage form for each day, as administration preferably proceeds once daily.

If the menstrual cycle is 28 days long, the kit according to the invention preferably comprises at least as many dosage forms containing trimegestone as are necessary for administering trimegestone on at least 21, preferably 24 successive days of the 28-day menstrual cycle. If trimegestone is administered on fewer than 28 days, for the remaining days up to the end of the 28 days of the menstrual cycle, the kit according to the invention may contain either no dosage forms at all, or preparations containing iron, preparations containing folic acid, folates, folinic acid, folinates or placebos, preferably a preparation containing iron. It is necessary here for at least one of the dosage forms containing trimegestone of the kit according to the invention to be a dosage form according to the invention as described above.

If the menstrual cycle is extended, i.e. is more than 28 days long, the number of dosage forms containing trimegestone contained in the kit according to the invention is correspondingly increased, wherein preferably again at least one of the dosage forms containing trimegestone is a dosage form according to the invention as described above.

In a preferred embodiment, the kit according to the invention comprises all the dosage forms containing trimegestone which are necessary for administering trimegestone for at least two, more preferably at least three, still more preferably at least four, most preferably at least five and in particular at least six menstrual cycles.

In a preferred embodiment, the kit according to the invention is designed for mono- or multiphasic administration of trimegestone in combination with an oestrogen, preferably ethinyloestradiol. The menstrual cycle is here preferably 28 days long. In the bi-, tri- and tetraphasic regimens, the daily dose of trimegestone and of the oestrogen is in each case constant on all days within a phase and different on two successive days of different phases.

Trimegestone is preferably used in the dosage forms in combination with ethinyloestradiol or in combination with ethinyloestradiol and oestradiol (17β-oestradiol).

Preferred embodiments no. 1, 2₁, 2₂, 3₁, 3₂, 3₃, 4₁ and 4₂ of the kit according to the invention comprise in total 21-25 dosage forms containing trimegestone, wherein, depending on the number of phases, these contain trimegestone in doses A1, A2, A3 and at least one oestrogen, preferably ethinyloestradiol, in dose B according to the following table:

| | | Number of phases | | | | | | | |
|---|---|---|---|---|---|---|---|---|---|
| | | 1 | 2 | 2 | 3 | 3 | 3 | 4 | 4 |
| Embodiment no. | | 1 | 2₁ | 2₂ | 3₁ | 3₂ | 3₃ | 4₁ | 4₂ |
| Total dosage forms | | 21-25 | 21-25 | 21-25 | 21-25 | 21-25 | 21-25 | 21-25 | 21-25 |
| 1 | Number of | 21-25 | 7-13 | 7-13 | 3-8 | 3-8 | 3-8 | 3-8 | 3-8 |
| | administration units | | | | | | | | |
| | Trimegestone dose | A1 | A2 | A1 | A2 | A2 | A1 | A2 | A2 |
| | Oestrogen dose | B | B | B | B | B | B | B | B |
| | (equivalent dose to | | | | | | | | |
| | ethinyloestradiol) | | | | | | | | |
| 2 | Number of | | 12-18 | 12-18 | 4-15 | 4-15 | 4-15 | 4-15 | 4-15 |
| | administration units | | | | | | | | |
| | Trimegestone dose | | A1 | A2 | A2 | A1 | A2 | A1 | A2 |
| | Oestrogen dose | | B | B | B | B | B | B | B |
| | (equivalent dose to | | | | | | | | |
| | ethinyloestradiol) | | | | | | | | |
| 3 | Number of | | | | 4-15 | 4-15 | 4-15 | 4-15 | 4-15 |
| | administration units | | | | | | | | |
| | Trimegestone dose | | | | A1 | A2 | A2 | A2 | A1 |
| | Oestrogen dose | | | | B | B | B | B | B |
| | (equivalent dose to | | | | | | | | |
| | ethinyloestradiol) | | | | | | | | |
| 4 | Number of | | | | | | | 2-5 | 2-5 |
| | administration units | | | | | | | | |
| | Trimegestone dose | | | | | | | A3 | A3 |
| | Oestrogen dose | | | | | | | B | B |
| | (equivalent dose to | | | | | | | | |
| | ethinyloestradiol) | | | | | | | | |

The particular ranges of values of the doses for the particular combinations of A1, A2, A3 and B for each one of these embodiments no. 1, 2₁, 2₂, 3₁, 3₂, 3₃, 4₁ and 4₂ may be found in the following tables, the dose B of the at least one oestrogen being stated as the equivalent dose to ethinyloestradiol:

| a | a | |
|---|---|---|
| A1 | >500 | μg |
| A2 | ≧40 | μg |
| A3 | ≧0 | μg |
| B | 5.0-55 | μg |

or

| b | b1 | | b2 | | b3 | | b4 | |
|---|---|---|---|---|---|---|---|---|
| | preferably | | more | | still more | | in particular | |
| | | | preferably | | preferably | | | |
| A1 | 510-990 | μg | 525-975 | μg | 550-950 | μg | 550-750 | μg |
| A2 | 40-990 | μg | 40-750 | μg | 120-750 | μg | 260-500 | μg |
| A3 | 0-990 | μg | 0-750 | μg | 0-500 | μg | 260-500 | μg |
| B | 5.0-55 | μg | 10-50 | μg | 20-45 | μg | 25-40 | μg |

or

| c | c1 | | c2 | | c3 | | c4 | |
|---|---|---|---|---|---|---|---|---|
| | preferably | | more | | still more | | in particular | |
| | | | preferably | | preferably | | | |
| A1 | 1,010-1,990 | μg | 1,025-1,975 | μg | 1,050-1,950 | μg | 1,050-1,750 | μg |
| A2 | 40-1,990 | μg | 40-750 | μg | 120-750 | μg | 260-500 | μg |
| A3 | 0-1,990 | μg | 0-750 | μg | 0-500 | μg | 260-500 | μg |
| B | 5.0-55 | μg | 7.5-50 | μg | 10-45 | μg | 15-40 | μg |

or

| d | d1 | | d2 | | d3 | | d4 | |
|---|---|---|---|---|---|---|---|---|
| | preferably | | more | | still more | | in particular | |
| | | | preferably | | preferably | | | |
| A1 | >2,000 | μg | >2,500 | μg | ≧3,000 | μg | ≧4,000 | μg |
| A2 | 40-5,000 | μg | 40-750 | μg | 120-750 | μg | 260-500 | μg |
| A3 | 0-5,000 | μg | 0-750 | μg | 0-500 | μg | 260-500 | μg |
| B | 5.0-55 | μg | 7.5-50 | μg | 10-45 | μg | 15-40 | μg |

The following preferred embodiments may be individualized: 1^{a}, 2₁^{a}, 2₂^{a}, 3₁^{a}, 3₂^{a}, 3₃^{a}, 4₁^{a} and 4₂^{a}; 1^{b1}, 2₁^{b1}, 2₂^{b1}, 3₁^{b1}, 3₂^{b1}, 3₃^{b1}, 4₁^{b1} and 4₂^{b1}; 1^{b2}, 2₁^{b2}, 2₂^{b2}, 3₁^{b2}, 3₂^{b2}, 3₃^{b2}, 4₁^{b2} and 4₂^{b2}; 1^{b3}, 2₁^{b3}, 2₂^{b3}, 3₁^{b3}, 3₂^{b3}, 3₃^{b3}, 4₁^{b3} and 4₂^{b3}; 1^{b4}, 2₁^{b4}, 2₂^{b4}, 3₁^{b4}, 3₃^{b4}, 4₁^{b4} and 4₂^{b4}; 1^{c1}, 2₁^{c1}, 2₂^{c1}, 3₁^{c1}, 3₂^{c1}, 3₃^{c1}, 4₁^{c1} and 4₂^{c1}; 1^{c2}, 2₁^{c2}, 2₂^{c2}, 3₁^{c2}, 3₂^{c2}, 3₃^{c2}, 4₁^{c2} and 4₂^{c2}; 1^{c3}, 2₁^{c3}, 2₂^{c3}, 3₁^{c3}, 3₂^{c3}, 3₃^{c3}, 4₁^{c3} and 4₂^{c3}; 1^{c4}, 2₁^{c4}; 2₂^{c4}, 3₁^{c4}, 3₂^{c4}, 3₃^{c4}, 4₁^{c4} and 4₂^{c4}; 1^{d1}, 2₁^{d1}, 2₂^{d1}, 3₁^{d1}, 3₂^{d1}, 3₃^{d1}, 4₁^{d1} and 4₂^{d1}; 1_{d2}, 2₁^{d2}, 2₂^{d2}, 3₁^{d2}, 3₂^{d2}, 3₃^{d2}, 4₁^{d2} and 4₂^{d2}; 1^{d3}, 2₁^{d3}, 2₂^{d3}, 3₁^{d3}, 3₂^{d3}, 3₃^{d3}, 4₁^{d3} and 4₂^{d3}; and 1^{d4}, 2₁^{d4}, 2₂^{d4}, 3₁^{d4}, 3₂^{d4}, 3³_{d4}, 4₁^{d4} and 4₂^{d4}.

A particularly preferred kit according to the invention contains all dosage forms that are necessary in order to allow for the administration of trimegestone in combination with ethinyloestradiol on 24 successive days of the menstrual cycle, thereby following any of regimens 1′, 2₁′, 2₂′, 3₁′, 3₂′, 3₃′, 4₁′ and 4₂′ as described above in connection with the method according to the invention.

Trimegestone, optionally in combination with an oestrogen and/or a further gestagen, may also be taken optionally for a period of more than 28 days for therapeutic reasons, such as for example for the treatment and/or prevention of at least one of the complaints or conditions selected from the group consisting of bleeding disorders; dysmenorrhoea; conditions dependent on the menstrual cycle, such as endometriosis, polycystic ovarian syndrome (PCOS), uterus myomatosus, functional cysts, premenstrual syndrome and headaches/migraine; conditions influenced by the menstrual cycle, such as epilepsy, multiple sclerosis, diabetes mellitus, depression, schizophrenia, asthma and Parkinson's disease; and androgen-induced disorders, such as seborrhoea, acne, androgenetic alopecia and hirsutism.

The present invention accordingly also relates to the use of trimegestone, optionally in combination with an oestrogen (such as ethinyloestradiol) and/or a further gestagen, for the production of a medicine (e.g. an oral contraceptive), preferably with a dose of trimegestone of more than 500 μg and preferably less than 2,000 μg, for the treatment and/or prevention of at least one of the complaints or conditions selected from the group consisting of bleeding disorders; dysmenorrhoea; conditions dependent on the menstrual cycle, such as endometriosis, polycystic ovarian syndrome (PCOS), uterus myomatosus, functional cysts, premenstrual syndrome (PMS), premenstrual dysphoric disorder (PMDD) and headaches/migraine; and androgen-induced disorders, such as seborrhoea, acne, androgenetic alopecia and hirsutism.

The dosage forms according to the invention may be prepared by conventional processes. The following examples are not to be considered as limiting the scope of the invention:

### EXAMPLE 1

| | |
|---|---|
| Per tablet | Per batch |

a) Composition

| | | |
|---|---|---|
| Ethinyloestradiol | 0.020 mg | 0.002 kg |
| Trimegestone | 2.000 mg | 0.200 kg |
| Povidone K30 | 3.000 mg | 0.300 kg |
| Lactose monohydrate | 31.980 mg | 3.198 kg |
| Maize starch | 12.000 mg | 1.200 kg |
| Magnesium stearate | 0.500 mg | 0.050 kg |
| Colloidal silicon dioxide | 0.500 mg | 0.050 kg |

b) Composition

| | | |
|---|---|---|
| Ethinyloestradiol | 0.015 mg | 0.0015 kg |
| Trimegestone | 2.000 mg | 0.2000 kg |
| Povidone K30 | 3.000 mg | 0.300 kg |
| Lactose monohydrate | 32.985 mg | 3.2985 kg |
| Maize starch | 12.000 mg | 1.2000 kg |
| Magnesium stearate | 0.500 mg | 0.0500 kg |
| Colloidal silicon dioxide | 0.500 mg | 0.0500 kg |

Ethinyloestradiol (EE) and povidone K30 (polyvinylpyrrolidone) are dissolved in 600 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and colloidal silicon dioxide and pressed on a tablet press with 5 mm punches into tablets with a weight of 50 mg.

The tablets of composition a) are coated with a hypromellose-based coating (e.g. Opadry YS-1-2184 made by Colorcon), coating composition 2 mg per tablet, and packaged into a packaging comprising 24 hormone-containing daily units and 4 hormone-free daily units with the same composition but without hormones.

The tablets of composition b) are coated with a hypromellose-based coating (e.g. Opadry YS-1-2184 made by Colorcon) of the following composition (coating composition 2 mg per tablet).

| Composition of the coating | | |
|---|---|---|
| Hypromellose | 6 mPas | 0.1351 kg |
| Polyethylene glycol | 6000 | 0.0395 kg |
| Propylene glycol | | 0.0054 kg |
| Purified water | | 1.6200 kg |

24 hormone-containing tablets and 4 hormone free tablets, each as daily dosage unit, are packed into one packaging.

### EXAMPLE 2

| | |
|---|---|
| Per tablet | Per batch |

a) Composition

| | | |
|---|---|---|
| Ethinyloestradiol | 0.015 mg | 0.0015 kg |
| Trimegestone | 3.000 mg | 0.3000 kg |
| Povidone K30 | 4.000 mg | 0.4000 kg |
| Lactose monohydrate | 63.485 mg | 6.3485 kg |
| Maize starch | 10.000 mg | 1.0000 kg |
| Magnesium stearate | 0.500 mg | |

b) Composition

| | | |
|---|---|---|
| Ethinyloestradiol | 0.025 mg | 0.0025 kg |
| Trimegestone | 5.000 mg | 0.5000 kg |
| Povidone K30 | 4.500 mg | 0.4500 kg |
| Lactose monohydrate | 59.975 mg | 5.9975 kg |
| Maize starch | 10.000 mg | 1.0000 kg |
| Magnesium stearate | 0.500 mg | 0.0500 kg |

Ethinyloestradiol (EE) and povidone K30 (polyvinylpyrrolidone) are dissolved in 950 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and pressed on a tablet press with 6 mm punches into tablets with a weight of 80 mg.

The tablets of composition a) are coated with a hypromellose-based coating (e.g. Opadry YS-1-2184 made by Colorcon), coating composition 2 mg per tablet, and packaged into a packaging comprising 24 hormone-containing daily units and 4 hormone-free daily units.

The tablets of composition b) are coated with a hypromellose-based coating (e.g. Opadry YS-1-2184 made by Colorcon) of the following composition (coating composition 1 mg per tablet).

| Composition of the coating | | |
|---|---|---|
| Hypromellose | 6 mPas | 0.068 kg |
| Polyethylene glycol | 6000 | 0.020 kg |
| Propylene glycol | | 0.002 kg |
| Purified water | | 1.810 kg |

24 hormone-containing tablets and 4 hormone free tablets, each as daily dosage unit, are packed into one packaging.

### EXAMPLE 3

2-Phase Contraceptive

a) Composition of the 1. phase

| | Per tablet |
|---|---|
| Ethinyloestradiol | 0.020 mg |
| Trimegestone | 2.000 mg |
| Povidone K30 | 3.000 mg |
| Lactose monohydrate | 31.980 mg |
| Maize starch | 12.000 mg |
| Magnesium stearate | 0.500 mg |
| Colloidal silicon dioxide | 0.500 mg |

Ethinyloestradiol (EE) and povidone K30 (polyvinylpyrrolidone) are dissolved in 600 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and colloidal silicon dioxide and pressed on a tablet press with 5 mm punches into tablets with a weight of 50 mg.

b) Composition of the 2. phase

As indicated under a), hormone-free, folic acid-containing tablets with a weight of 50 mg are produced, wherein the sodium salt of the folic acid is dissolved in 600 ml of aqueous ethanol.

| | Per tablet |
|---|---|
| Ethinyloestradiol | 0.020 mg |
| Trimegestone | 3.000 mg |
| Povidone K30 | 3.000 mg |
| Lactose monohydrate | 31.000 mg |
| Maize starch | 12.000 mg |
| Magnesium stearate | 0.500 mg |
| Colloidal silicon dioxide | 0.500 mg |

Some tablets are produced as disclosed under a)

The tablets under a) and b) are coated with a hypromellose-based coating (e.g. Opadry YS-1-2184 made by Colorcon), coating composition 2 mg per tablet. 12 hormone-containing daily units produced according to a) and 12 hormone-containing daily units produced according to b) and 4 hormone-free daily units are packed in a package marked for a daily administration.

### EXAMPLE 4

| | a) | b) |
|---|---|---|
| Composition | Per tablet | Per tablet |
| Ethinyloestradiol | 0.020 mg | |
| Trimegestone | 2.000 mg | |
| Sodium folate | 0.050 mg | 3.000 mg |
| Povidone K30 | 3.000 mg | 3.000 mg |
| Lactose monohydrate | 31.930 mg | 31.000 mg |
| Maize starch | 12.000 mg | 12.000 mg |
| Magnesium stearate | 0.500 mg | 0.500 mg |
| Colloidal silicon dioxide | 0.500 mg | 0.500 mg |

a) Ethinyloestradiol (EE) and povidone K30 (polyvinylpyrrolidone) and sodium folate are dissolved in 600 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and colloidal silicon dioxide and pressed on a tablet press with 5 mm punches into tablets with a weight of 50 mg.

b) As set forth under a), hormon-free, folic acid containing tablet having a weight of 50 mg are prepared by dissolving sodium folate in 600 ml aqueous ethanol.

The tablets a) and b), respectively, are coated with a coating based on hypromellose (e.g. Opadry YS-1-2184, Colorcon); coating composition 2 mg per tablet.

21 hormone-containing daily units produced according to a) and 7 hormone-free daily units produced according to b) are packed in a package marked for a daily administration.

### EXAMPLE 5

120 tablets according to Example 1 a) are packed in a blister and marketed for daily administration on 120 successive days.

### EXAMPLE 6

| Composition | Per tablet |
|---|---|
| Ethinyloestradiol | 0.030 mg |
| Trimegestone | 2.000 mg |
| Povidone K30 | 3.000 mg |
| Lactose monohydrate | 31.970 mg |
| Maize starch | 12.000 mg |
| Magnesium stearate | 0.500 mg |
| colloidal silicon dioxide | 0.500 mg |

Ethinyloestradiol (EE) and povidone K30 (polyvinylpyrrolidone) are dissolved in 600 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and colloidal silicon dioxide and pressed on a tablet press with 5 mm punches into tablets with a weight of 50 mg.

The tablets are coated with a hypromellose-based coating of the following composition (coating composition 2 mg per tablet):

| Composition of the coating | | |
|---|---|---|
| Hypromellose | 6 mPas | 0.1351 kg |
| Polyethylene glycol | 6000 | 0.0395 kg |
| Propylene glycol | | 0.0054 kg |
| Purified water | | 1.6200 kg |

The tablets are packed into a blister containing 189 daily units and marketed for daily administration on 189 successive days.

### EXAMPLE 7

| Composition | Per tablet |
|---|---|
| Ethinyloestradiol | 0.015 mg |
| Trimegestone | 2.000 mg |
| Povidone K30 | 4.000 mg |
| Lactose monohydrate | 63.485 mg |
| Maize starch | 10.000 mg |
| Magnesium stearate | 0.500 mg |

Ethinyloestradiol (EE) and povidone K30 (polyvinylpyrrolidone) are dissolved in 950 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and pressed on a tablet press with 6 mm punches into tablets with a weight of 80 mg.

The tablets are coated with a hypromellose-based coating of the following composition (coating composition 2 mg per tablet):

| Composition of the coating | | |
|---|---|---|
| Hypromellose | 6 mPas | 0.1351 kg |
| Polyethylene glycol | 6000 | 0.0395 kg |
| Propylene glycol | | 0.0054 kg |
| Purified water | | 1.6200 kg |

The tablets are packed into a blister containing 365 daily units and are marketed for daily administration on 365 successive days.

### EXAMPLE 8

| Composition | Per tablet |
|---|---|
| Ethinyloestradiol | 0.030 mg |
| Trimegestone | 5.000 mg |
| Povidone K30 | 4.500 mg |
| Lactose monohydrate | 60.470 mg |
| Maize starch | 10.000 mg |
| Magnesium stearate | 0.500 mg |

Ethinyloestradiol (EE) and povidone K30 (polyvinylpyrrolidone) are dissolved in 950 ml of ethanol. Trimegestone (particle size 90% <50 μm), lactose and maize starch are mixed in a mixer/pelletiser (Diosna P25) for 5 mins and then moistened thoroughly and mixed with the ethanolic EE/PVP solution. The moist composition is forced through a 3 mm screen and dried in a vacuum drying cabinet. The dried granular product is disagglomerated through a 0.6 mm screen, mixed with magnesium stearate and pressed on a tablet press with 6 mm punches into tablets with a weight of 80 mg.

The tablets are coated with a hypromellose-based coating of the following composition (coating composition 1 mg per tablet):

| Composition of the coating | | |
|---|---|---|
| Hypromellose | 6 mPas | 0.068 kg |
| Polyethylene glycol | 6000 | 0.020 kg |
| Propylene glycol | | 0.002 kg |
| Purified water | | 0.810 kg |

The tablets are packed into a blister containing 150 daily units and are marketed for daily administration on 150 successive days.

1. A method for contraception comprising administering a contraceptively effective amount of trimegestone to a woman of child-bearing age on at least 21 successive days, beginning on day 1 of the menstrual cycle, wherein on at least one of the at least 21 successive days the daily dose of trimegestone is more than 500 μg.

2. The method according to claim 1, wherein on at least one of the at least 21 successive days the daily dose of trimegestone is
in the range from more than 500 μg to less than 2,000 μg; or
more than 2,000 μg.

3. The method according to claim 1, wherein the trimegestone is administered orally.

4. The method according to claim 1, wherein the menstrual cycle is 28 days long or longer than 28 days.

5. The method according to claim 1, wherein trimegestone is administered in combination with at least one oestrogen at least on one of the at least 21 successive days.

6. The method according to claim 5, wherein the oestrogen is selected from the group consisting of chlorotrianisene, dienestrol, diethylstilbestrol, oestradiol, oestriol, oestrone, ethinyloestradiol, hexoestrol, mestranol, methallenoestril, methyloestrenol, promestriene and conjugated oestrogens and the pharmaceutically acceptable esters thereof.

7. The method according to claim 5, wherein the daily dose of the oestrogen corresponds to an equivalent dose of 5.0 to 55 μg of ethinyloestradiol.

8. The method according to claim 6, wherein ethinyloestradiol is administered in a daily dose of 1.0 to 50 μg and/or oestradiol is administered in a daily dose of 1,000 to 10,000 μg at least on one of the at least 21 successive days.

9. The method according to claim 1, wherein at least on one of the at least 21 successive days trimegestone
is administered either not together with oestradiol
or together with a combination of oestradiol and ethinyloestradiol.

10. The method according to claim 1, wherein the daily dose of trimegestone is identical on each of the at least 21 successive days.

11. The method according to claim 1, wherein trimegestone is not administered on all the days of the menstrual cycle and that, on the days which follow the least 21 successive days,
a placebo is administered, a preparation containing iron is administered,
a preparation containing folic acid, folinic acid and/or a salt thereof is administered,
a preparation containing an oestrogen is administered, or
nothing at all is administered.

12. The method according to claim 1, wherein trimegestone is administered in combination with at least one further gestagen at least on one of the at least 21 successive days.

13. The method according to claim 12, wherein the further gestagen is selected from the group consisting of allyloestrenol, chlormadinone, danazol, demegestone, desogestrel, dienogest, drospirenone, dydrogesterone, ethisterone, etynodiol, gestodene, gestonorone, hydroxyprogesterone, levonorgestrel, lynoestrenol, medroxyprogesterone, medrogestone, megestrol, methyloestrenol, methylnortestosterone, nomegestrol, norethisterone, norethynodrel, norgestrel, norgestimate, progesterone, promegestone and tibolone, and the pharmaceutically acceptable esters thereof.

14. The method according to claim 12, wherein the daily dose of the further gestagen corresponds to an equivalent dose of 100 to 5,000 μg of chlormadinone acetate.

15. The method according to claim 1, which is carried out for least 6 successive menstrual cycles.

16. A pharmaceutical composition comprising trimegestone in a quantity of more than 500 μg in combination with ethinyloestradiol and pharmaceutically acceptable adjarents.

17. A pharmaceutical composition comprising trimegestone in a quantity of
more than 500 μg and less than 1,000 μg; or
more than 1,000 μg and less than 2,000 μg; or
more than 2,000 μg.

18. An oral dosage form of the pharmaceutically composition of claim 16 comprising trimegestone in a quantity of more than 500 μg, wherein the dosage form is selected from the group consisting of film coated tablets, sugar coated tablets and capsules.

19. The composition according to claim 17 or the oral dosage form according to claim 18, additionally containing at least one oestrogen.

20. The composition or dosage form according to claim 19, wherein the oestrogen is selected from the group consisting of chlorotrianisene, dienestrol, diethylstilbestrol, oestradiol, oestriol, oestrone, ethinyloestradiol, hexoestrol, mestranol, methallenoestril, methyloestrenol, promestriene and conjugated oestrogens or the pharmaceutically acceptable esters thereof.

21. The composition dosage form according to claim 19, wherein the quantity of the oestrogen corresponds to an equivalent dose of 5.0 to 55 μg of ethinyloestradiol.

22. The composition according to claim 16, which contains either no oestradiol, or which contains oestradiol in combination with ethinyloestradiol.

23. The composition according to claim 16, additionally containing at least one further gestagen.

24. The composition dosage form according to claim 23, wherein the gestagen is selected from the group consisting of allyloestrenol, chlormadinone, danazol, demegestone, desogestrel, dienogest, drospirenone, dydrogesterone, ethisterone, etynodiol, gestodene, gestonorone, hydroxyprogesterone, levonorgestrel, lynoestrenol, medroxyprogesterone, medrogestone, megestrol, methyloestrenol, methylnortestosterone, nomegestrol, norethisterone, norethynodrel, norgestrel, norgestimate, progesterone, promegestone and tibolone, the pharmaceutically acceptable esters thereof.

25. The composition or dosage form according to claim 24, wherein the quantity of the gestagen corresponds to an equivalent dose of 100 to 5,000 μg of chlormadinone acetate.

26. The composition according to claim 16, additionally containing folic acid, folinic acid and/or a salt thereof.

27. The composition or dosage form according to claim 26, wherein the content of the folic acid, folinic acid and/or salt thereof is within the range of from 0.1 to 7.5 mg.

28. The composition according to claim 16, additionally containing one or more auxiliary substances independently selected from the group comprising salt formers, buffers, emulsifiers, solubilisers, wetting agents, antifoaming agents, gel formers, thickeners, film formers, surfactants, binders, slip agents, lubricants, embedding agents, mould release agents, flow-control agents, disintegrants, chelating agents, sorbents, fillers, pharmaceutical solvents, antioxidants, preservatives, plasticizers, flavor and odor correctives and colorants.

29. The composition according to claim 16 for contraception.

30. A kit comprising at least one composition according to claim 16.

31. The kit according to claim 30, which comprises in total 21±5 dosage forms for once daily administration on 21±25 successive days, each of which contains an oestrogen in a quantity corresponding to an equivalent dose of 5.0 to 55 μg of ethinyloestradiol and trimegestone in a quantity of more than 500 μg.

32. The kit according to claim 30, which comprises in total 21±25 dosage forms, 7±13 of which are for once daily administration during an initial phase of 7±13 successive days and 12-18 of which for once daily administration during a final phase of 12-18 successive days uninterruptedly following the initial phase, wherein each dosage form contains an oestrogen in a quantity corresponding to an equivalent dose of 5.0 to 55 μg of ethinyloestradiol, and
the 7±13 dosage forms for administration during the initial phase contain trimegestone in a quantity of at least 40 μg each and the 12±18 dosage forms for administration during the final phase contain trimegestone in a quantity of more than 500 μg each; or
the 7±13 dosage forms for administration during the initial phase contain trimegestone in a quantity of more than 500 μg each and the 12±18 dosage forms for administration during the final phase contain trimegestone in a quantity of at least 40 μg each;
with the proviso that the quantity of trimegestone contained in the 7±13 dosage forms for administration during the initial phase differs from the quantity of trimegestone contained in the 12±18 dosage forms for administration during the final phase.

33. The kit according to claim 30, which comprises in total 21 to 25 dosage forms, 3 to 8 of which for once daily administration during an initial phase of 3-8 successive days, 4 to 15 of which for once daily administration during an intermediate phase of 4 to 15 successive days uninterruptedly following the initial phase, and 4 to 15 of which for once daily administration during a final phase of 4 to 15 successive days uninterruptedly following the intermediate phase, wherein each dosage form contains an oestrogen in a quantity corresponding to an equivalent dose of 5.0 to 55 μg of ethinyloestradiol, and
the 3-8 dosage forms for administration during the initial phase contain trimegestone in a quantity of at least 40 μg each, the 4 to 15 dosage forms for administration during the intermediate phase contain trimegestone in a quantity of at least 40 μg each, and the 4 to 15 dosage forms for administration during the final phase contain trimegestone in a quantity of more than 500 μg each; or
the 3-8 dosage forms for administration during the initial phase contain trimegestone in a quantity of at least 40 μg each, the 4 to 15 dosage forms for administration during the intermediate phase contain trimegestone in a quantity of more than 500 μg each, and the 4 to 15 dosage forms for administration during the final phase contain trimegestone in a quantity of at least 40 μg each; or
the 3-8 dosage forms for administration during the initial phase contain trimegestone in a quantity of more than 500 μg each, the 4 to 15 dosage forms for administration during the intermediate phase contain trimegestone in a quantity of at least 40 μg each, and the 4 to 15 dosage forms for administration during the final phase contain trimegestone in a quantity of at least 40 μg each;
with the proviso that the quantity of trimegestone contained in the 3 to 8 dosage forms for administration during the initial phase differs from the quantity of trimegestone contained in the 4-15 dosage forms for administration during the intermediate phase, which in turn differs from the quantity of trimegestone contained in the 4 to 15 dosage forms for administration during the final phase.

34. The kit according to claim 30, which comprises in total more than 28 dosage forms for once daily administration on successive days, at least one of which dosage forms contains an oestrogen in a quantity corresponding to an equivalent dose of 5.0 to 55 μg of ethinyloestradiol and trimegestone in a quantity of more than 500 μg.

35. The kit according to claim 34, which comprises at least 120 dosage forms for once daily administration on 120 successive days, each of which dosage forms contains an oestrogen in a quantity corresponding to an equivalent dose of 5.0 to 55 μg of ethinyloestradiol and trimegestone in a quantity of more than 500 μg.
